(19) United States
(12) Patent Application Publication
(10) Pub. No.: US 2020/0297326 A1
(43) Pub. Date: Sep. 24, 2020

(54) TESTING SYSTEM FOR ULTRASONIC IMAGING SYSTEM

(71) Applicant: ACERTARA ACOUSTIC LABORATORIES LLC, Longmont, CO (US)

(72) Inventors: Levi Moore, Longmont, CO (US); Robert Brown, Longmont, CO (US); Connor Timms, Longmont, CO (US)

(21) Appl. No.: 16/358,328

(22) Filed: Mar. 19, 2019

(51) Int. Cl. A61B 8/00; G01N 29/44; G01N 29/30

(52) U.S. Cl. CPC A61B 8/587; G01N 29/4427; G09B 23/286; G01N 2291/02475; G01N 29/30

(57) ABSTRACT

A system for use in testing ultrasound systems that includes a tissue mimicking phantom and computer-readable instructions that are configured to automatically compare images of the phantom obtained by the ultrasound system to one or more reference images (e.g., indicating how the obtained images should appear) and provide output that assists personnel in assessing the operationality of the ultrasound system. Any appropriate digital recording device may be used to digitally store signals received from outputs of the ultrasound system for use by the image comparison module on a real-time basis or after full acquisition of the phantom images.

### FIELD OF THE INVENTION

This application generally relates to ultrasound systems and, more particularly, to the testing of ultrasound systems.

### BACKGROUND OF THE INVENTION

Transmission of pressure waves such as acoustic radiation toward a target and reception of the scattered radiation may be managed by a modern acoustic-imaging system, which may take a variety of forms. For instance, acoustic imaging is an important technique that may be used at different acoustic frequencies for varied applications that range from medical imaging to nondestructive testing of structures. The techniques generally rely on the fact that different structures have different acoustic impedances, allowing characterization of structures and their interfaces from information embodied by the different scattering patterns that result. While most applications use radiation reflected from structures, some techniques also make use of information in transmitted patterns.

For example, many modern systems are based on multiple-element array transducers that may have linear, curved-linear, phased-array or similar characteristics, and which may be embodied in an acoustic probe. Summing the contributions of the multiple transducer elements comprised by a transducer array allows images to be formed. It is sometimes desired to analyze certain portions of received pressure waves relative to other portions of pressure waves. In the case of ultrasound probes, for instance, the failure of a small number of elements in a given array, or a few defective receive channels in the acoustic system itself, may not be readily perceptible to users because of the averaging effect of summing many elements to form an acoustic beam. But the failure of even a small number of elements or receive channels can significantly degrade the performance of acoustic imaging systems, notably in certain modes of operation like those known as “Doppler” or “near-field” imaging modes.

Materials which closely mimic the ultrasonic propagation characteristics of human tissue are employed in imaging “phantoms” for use in testing ultrasound systems. These phantoms may be used to carry out performance checks on ultrasound scanners. Phantoms may also be used for training or testing student technologists in the operation of ultrasound scanners or the interpretation of ultrasound images produced by such scanners. For instance, ultrasound phantoms embodying the desired features for mimicking soft tissue may be prepared from a mixture of gelatin, water, n-propanol and graphite powder, with a preservative; a mixture of oil and gelatin; or the like. The mixture may be admitted into a container in such a way as to exclude air bubbles from forming in the container. In addition to the tissue mimicking material itself, scattering particles, spaced sufficiently close to each other such that an ultrasound scanner is incapable of resolving individual scattering particles, and testing spheres or other targets (e.g., to simulate in situ structures within the human body), may be located within the phantom container (e.g., suspended in the tissue mimicking material body).

For example, it is often desirable to have zones within a phantom which mimic the ultrasound characteristics of vessels, cysts or tumors found in the human body. To this end, thin walled, semi-rigid plastic tubing can be inserted within the foam material to mimic the ultrasound characteristics of vessels or sacs. Such an ultrasound phantom is useful in evaluating the ability of ultrasound medical diagnostic scanners to resolve target objects of selected sizes located throughout the tissue mimicking material. The objective is for the ultrasound scanner to accurately resolve the testing spheres or other targets from the background material and scattering particles.

In use, a testing technician (e.g., biomedical engineer) may initially grasp a probe of an ultrasound system and then do a free-hand alignment of the probe to the targets contained within the phantom. The ultrasound system may then be operated to obtain one or more images of the inside of the phantom and such images may be visually compared by the technician to one or more reference images (e.g., printed on outside of the phantom) to determine whether the probe is working well enough for use on a patient.

### SUMMARY

Existing manners of testing ultrasound systems with tissue mimicking phantoms are largely dependent on the biomedical engineer's (or other highly skilled technician's) ability to accurately align the probe in various manners (e.g., in relation to location on the phantom; pitch, roll, and yaw; etc.) with one or more particular areas on the phantom and to compare the obtained images with the reference image(s). In addition to the inherent subjectivity present in this arrangement, however, the number of people available to conduct such testing is limited as such technicians are typically required to possess biomedical engineering degrees or equivalent. Additionally, reference image(s) printed on the outside of the phantom are generic representations of the intended target placement and content and thus deviations in manufacturing can create misalignment between the image and the actual contents. Still further, detailed records of the images obtained during testing and comparison results are typically not produced; very often, stored records merely include printed screen captures from the ultrasound system placed into a physical storage location.

In view of at least the foregoing, the inventors have determined that an objective solution that is repeatable by multiple users with reduced alignment requirements and with digital record storage is needed. Broadly, disclosed herein is a system for use in testing ultrasound systems that includes a tissue mimicking phantom and computer-readable instructions that are configured to automatically compare (e.g., on a pixel by pixel basis) images of the phantom obtained by the ultrasound system to one or more reference images (e.g., indicating how the obtained images should appear) and provide output that assists personnel in assessing the accuracy or correctness of the ultrasound system. Modern manufacturing techniques may be used to place precise structures inside of the phantom to allow for both passive and active evaluation of the probe and its constituent pieces in a manner previously unattainable. Known structure geometry, measured probe output, and injected signals of a known magnitude may be incorporated.

In one arrangement, any appropriate digital recording device may be used to digitally store signals received from one or more outputs of the ultrasound system (e.g., SVGA, HDMI, etc.) for use by an image comparison or analysis module on a real-time basis or after full acquisition of the phantom images. In one variation, the phantom may include a mechanically keyed probe-specific probe holder that will allow the rapid and precise alignment between the probe and the phantom. More specifically, the holder may be positioned relative to the surface of the phantom and the various structures inside the phantom such that when the probe is seated in the holder, the probe is automatically positioned in an optimal manner relative to the phantom for use in obtaining images thereof.

Advantages of the disclosed system include substantial removal of the inherent subjectivity in existing manners of obtaining tissue mimicking phantom measurements, an increase in user to user testing comparability and in the pool of available testing personnel, a reduction in the number of false-positive and false-negative test results that are caused by faulty equipment, the digitization of testing records to create opportunities for testing to be performed quickly on a daily or even a case-by-case basis.

In one aspect, a method for use in assessing a performance of an ultrasound system includes receiving, at a processor from an ultrasound system under test, one or more input images of a tissue mimicking phantom, the image(s) containing a digital representation of the tissue mimicking phantom; determining one or more characteristic data values from the one or more input images; automatically identifying, by the processor, one or more corresponding respective reference data values from a database of reference data based on previously-obtained reference images of the phantom; analyzing, by the processor, the one or more characteristic data values in view of the one or more corresponding respective reference data values; and generating, by the processor, result data based on the analyzing step, wherein the result data indicates a performance of the ultrasound system under test.

In another aspect, a system, includes a tissue mimicking phantom including a plurality of objects disposed within a tissue mimicking material; an ultrasound system including a) an ultrasonic probe that is configured to generate and receive ultrasonic waves reflect from the plurality of objects and b) an imaging console that is configured to process the received ultrasonic waves to generate digitized image signals; and a testing controller that is configured to process characteristic data describing the digitized image signals against corresponding characteristic data describing one or more corresponding reference image signals to generate result data indicative of a performance of the ultrasound system.

A further understanding of the nature and advantages of the present invention may be realized by reference to the remaining portions of the specification and the drawings, wherein like reference labels are used through the several drawings to refer to similar components. In some instances, reference labels are followed with a hyphenated sublabel; reference to only the primary portion of the label is intended to refer collectively to all reference labels that have the same primary label but different sublabel s.

### BRIEF DESCRIPTION OF THE DRAWINGS

FIG. 1 is a block diagram of an ultrasonic imaging system.

FIG. 2 is a perspective view of an imaging phantom according to an embodiment.

FIG. 3 is a perspective view of another imaging phantom according to an embodiment.

FIG. 4 is a schematic diagram including a testing system for use in testing performance of an ultrasonic imaging system with an imaging phantom.

FIG. 5 is a schematic diagram of a map of reference image objects for use with the testing system of FIG. 4.

FIG. 6 is a schematic diagram of one of the reference image objects of FIG. 5.

### DETAILED DESCRIPTION

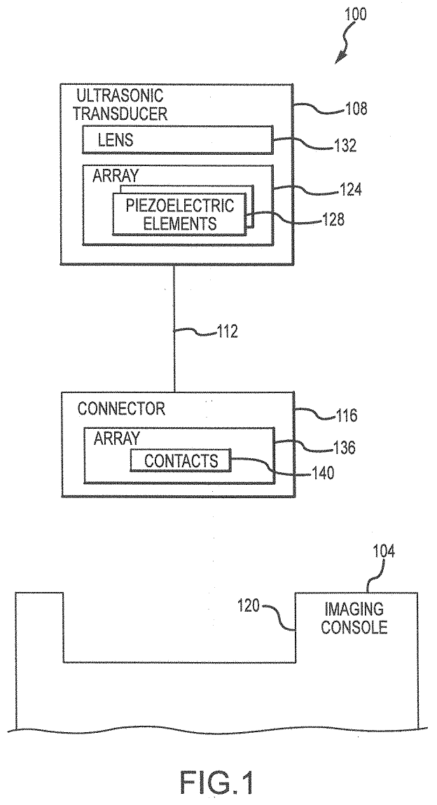

Disclosed herein is a system for testing performance characteristics of ultrasound systems that substantially removes the inherent subjectivity in existing manners of obtaining tissue mimicking phantom measurements, increases the pool of available testing personnel, reduces the number of false-positive and false-negative test results that are caused by faulty equipment, and digitizes testing records to create opportunities for testing to be performed quickly on a daily or even a case-by-case basis. Before discussing the testing system in more detail, reference is made to FIG. 1 which presents a block diagram of one type of ultrasonic imaging system 100 with which the testing system disclosed herein may be utilized. Broadly, the system 100 may include an imaging console 104 and an ultrasonic transducer 108 (e.g., transducer head) that is electrically interconnectable to the imaging console 104 by any appropriate cable assembly 112 and a connector or connector assembly 116, where the connector assembly 116 is configured to interface with a corresponding port 120 on the imaging console 104. The imaging console 104 may transmit a drive signal to the ultrasonic transducer 108 to cause piezoelectric elements 128 of the ultrasonic transducer 108 to transmit acoustic waves (e.g., ultrasound, ultrasonic waves) to a subject. The ultrasonic transducer 108 may be configured to receive reflection waves reflected by the interior of the subject and pass the same to the imaging console 104 for generation of one or more corresponding images. The ultrasonic transducer 108, cable assembly 112 and connector 116 may be referred to as an “acoustic probe,” “ultrasonic probe” or “ultrasound transducer.”

The ultrasonic transducer 108 may include any appropriate array 124 of piezoelectric elements 128 (e.g., linear, curved linear, etc.) that transmit ultrasonic waves towards a subject area, where summing the contributions of the multiple piezoelectric elements 128 allows images to be formed by the console 104 or other computer system. The ultrasonic transducer 108 may also include any appropriate acoustic lens 132 (e.g., layer of rubber-like material) that covers the array 124 to provide electrical safety, acoustic focusing, impedance matching, disinfection, and sealing of the ultrasonic transducer 108. While not shown, the ultrasonic transducer 108 may also include one or more other components such as backing layers, electrical contacts, and the like. The connector assembly 116 may include any appropriate housing (e.g., shield, casing, etc.) as well as an array 136 of electrical contacts 140 (e.g., pins, pads, flat surfaces, etc.) that are configured to electrically connect the multiple piezoelectric elements 128 to the imaging console 104.

Broadly, the imaging console 104 may be in the form of a housing including any appropriate arrangement of circuitry, components, and the like to receive inputs, generate corresponding drive signals to be transmitted to the piezoelectric elements 128 of the ultrasonic transducer 108 over cable assembly 112 and via the respective contacts 140 of the connector assembly 116 electrically interfaced with the imaging console 104. For instance, the imaging console 104 may include a control section (not shown) including any appropriate arrangement of processing units (e.g., processing cores, CPUs, etc.), memory (e.g., volatile memory such as random access memory or the like), storage (e.g., non-volatile such as hard disk, flash, etc.), etc. for purposes of operating each section of the ultrasonic imaging system 100 in conjunction with one or more developed programs or code portions (e.g., by way of the processing unit(s) executing one or more computer readable instruction sets in memory). The imaging console 104 may also include (or be in connection with) any appropriate operational input section (e.g., including switches, buttons, keyboard, etc.) in communication with the control section, a transmission section (e.g., circuitry) configured to transmit drive signals to the ultrasonic transducer 108 based on signals received from the control section, a receiving section (e.g., circuitry) configured to receive ultrasound reception signals under control of the control section, and one or more displays configured to display ultrasonic images of the subject under control of the control section. Various additional details of the imaging console 104 have been omitted from this discussion in the interest of brevity.

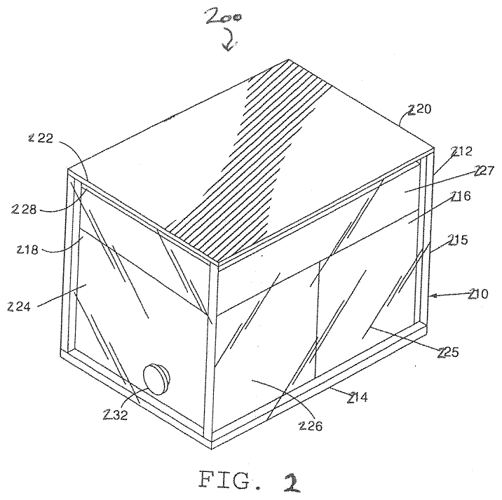

As discussed herein, tissue mimicking phantoms are often utilized as part of testing various performance characteristics of ultrasound and other imaging systems. In this regard, FIG. 2 presents one example of a phantom 200 with which the testing system disclosed herein may be utilized. Broadly, the phantom 200 includes a container 212 having a bottom 214 and walls 215 such as opposed faces 216 and opposed ends 218 to generally form a hollow, box-like container structure. Margins of the walls 215 remote from the bottom 214 may define a window 220 that may be closed with an ultrasound-transmitting window cover 222 made of any appropriate cohesive ultrasound transmitting material of suitable physical durability.

A body 224 of any appropriate tissue-mimicking material(s) may generally fill the container 212 up to the level of the window 220. In one arrangement, the body 224 may include several distinct sections 225, 226, and 227 of tissue-mimicking material to mimic the ultrasound properties of several corresponding body tissues. Although the sections 225, 226, and 227 are illustrated as rectangular blocks in contact with each other, they may also be formed of other shapes, such as shapes simulating human body structures. While not shown, various structures (e.g., tubing, spheres, etc.) may be positioned within the tissue-mimicking material(s) in various manners to simulate internal structures of the human body that may interact with and reflect transmitted ultrasonic waves for use in testing the performance of an ultrasound system (e.g., that of FIG. 1). While also not shown in FIG. 2, one or more reference images indicative of such internal structures may be printed on an outside of the phantom 200 or otherwise made available (e.g., on a display screen) for use by testing personnel in analyzing images of the internal structures obtained by the ultrasound system.

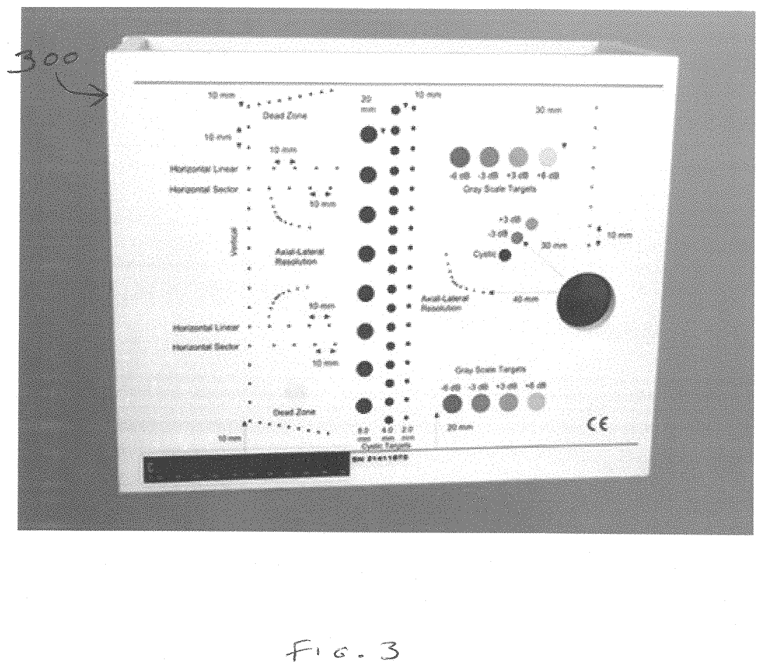

FIG. 3 presents another embodiment of an imaging phantom 300 that includes one or more reference images printed on an outside thereof to assist testing personnel in analyzing received ultrasound images. Various quality assurance and/or quality control “B-mode” (two-dimensional) parameters may be measured such as but not limited to image uniformity; depth of penetration; axial, lateral and elevational resolution; near field/dead zone; lesion detectability; high contrast (e.g., anechoic objects); low contrast (e.g., gray scale objects); and the like. Three-dimensional parameters (e.g., volume, reconstruction accuracy, etc.) and doppler parameters (e.g., flow rate, system sensitivity, directional discrimination, location of flow, maximum penetration, etc.) may also be measured. In any case, one or more of such measurements obtained by the ultrasound system may be compared to one or more corresponding reference measurements or ranges to determine whether the ultrasound system is operating in an acceptable manner.

As noted herein, existing manners of testing performance of ultrasound systems using phantoms require skilled technicians (e.g., biomedical engineers) to subjectively align the ultrasonic transducer with one or more particular portions on the phantom, obtain corresponding images of the interior of the phantom (e.g., on a display of or interconnected with the ultrasound system), and then visually compare the obtained images to one or more reference images physically printed onto the phantom (e.g., reference images 304 in FIG. 3) to determine whether the probe is working well enough for use on a patient. However, these procedures are highly dependent on the biomedical engineer's (or other highly skilled technician's) ability to accurately align the probe in various manners (e.g., in relation to location on the phantom; pitch, roll, and yaw; etc.) with one or more particular areas on the phantom and to compare the obtained images with the reference image(s) which can introduce uncertainty into the determined results, among other shortcomings.

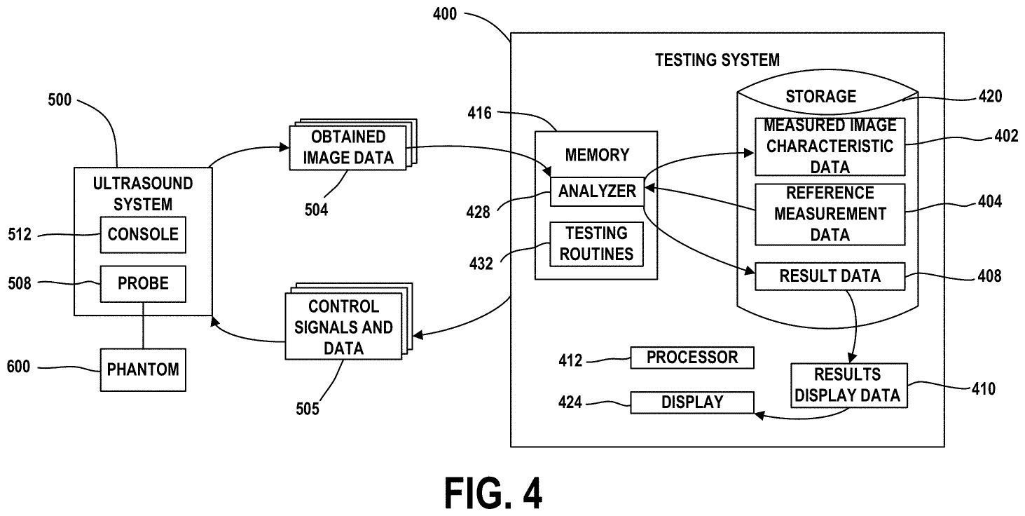

In this regard, FIG. 4 presents a schematic diagram of a testing system 400 that may be used to receive digitized image signals 504 from an ultrasound system 500 (e.g., system 100 of FIG. 1) and analyze the received digitized image signals 504 in view of reference data 404 to automatically generate result data 408 that conveys various performance characteristics of the ultrasound system 500. The testing system 400 may broadly be in the form of one or more computing devices or the like that include(s) a processor 412 (e.g., one or more processing cores, CPUs, etc.), memory 416 (e.g., volatile memory such as random access memory or the like), storage 420 (e.g., non-volatile such as hard disk, flash, etc.), a display 424, and the like, among other components that are not illustrated in the interest of brevity.

A set of any appropriate reference measurement data 404 specific to the particular phantom 600 being utilized may be initially obtained and stored in storage 420 in any appropriate manner (e.g., csv, table, relational database, etc.). For instance, a known “acceptable” probe 508 (e.g., a probe that is known in any appropriate manner to be functioning properly) may be initially used to obtain one or more reference images of the phantom 600 in any appropriate manner. From the reference image(s), one or more various types of reference measurement data 404 may be determined (e.g., calculated, deduced) such as pixel intensity, edge detection, image uniformity, image differential analyses, contrast and brightness, cross-sectional comparative analyses, and/or the like.

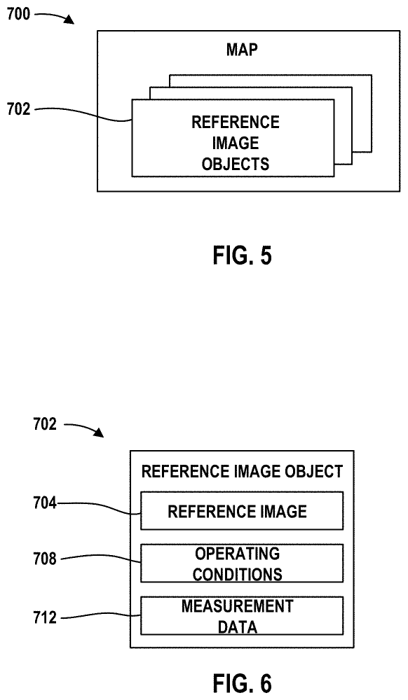

In one arrangement, a plurality of reference images of the phantom 600 may be obtained for generating a “map” 700 (e.g., see FIGS. 5 and 6) of reference image objects 702 (e.g., data structures) against which subsequently obtained images of the phantom 600 with probes 508 to be tested may be compared for use in determining the suitability of such probes 508 as discussed in more detail below. For instance, an operator may be initially instructed by the system 400 to obtain a plurality of reference images 704 of the phantom 600 from numerous (e.g., dozens, hundreds, etc.) points or locations about the phantom 600 and/or under a variety of other operating conditions as part of generating the map 700. Examples of operating conditions may include one or more of physical orientations and attitudes between the probe 508 of the ultrasound system and the phantom 600, ultrasound frequency wavelength, ultrasound intensity, ultrasound time-domain characteristics, ultrasound frequency-domain characteristics, and signal processing methodologies.

For each reference image 704 in the map 700, the analyzer 428 may be configured to identify the particular set 708 of operating conditions under which the reference image was obtained as well as the reference measurement data 712 (e.g., 404 from FIG. 4) corresponding to the reference image 704 and then store or otherwise associate the same as a respective reference image object 702. In the case where the reference image 704, its respective set 708 of operating conditions, and its respective reference measurement data 712 are stored in different respective portions of the storage 420 or memory 416, such data portions may be respectively indexed or linked by way of keys, identifiers, and/or the like for access by the analyzer 428. For instance, in the case where the analyzer identifies the set 712 of operating conditions of a particular reference image object 702 in a particular analysis, the analyzer 428 may have ready access to the corresponding reference measurement data 712 associated with the identified set 712 of operating conditions.

To test a probe 508 of the ultrasound system 500, a testing technician (e.g., biomedical engineer) may position the probe 508 over a scanning surface of the phantom 600 (e.g., over window 220 of phantom 200 of FIG. 2) and obtain one or more images (e.g., each in the nature of a digital representation of one or more portions of the phantom 600) of various targets contained within the phantom 600. The digitized image signals 504 generated by a console 512 of the ultrasound system 500 may then be transmitted in any appropriate manner to the testing system 400 whereupon the received signals 504 may be analyzed to generate the result data 408.

As shown, the testing system 400 may include one or more testing routines 432 that are broadly configured to dictate how the ultrasound system 500 is to be operated during the scanning of the phantom 600. In one arrangement, the testing routines 432 may be in the nature of a set of instructions that may be presented on the display 424 and that indicate to the testing technician one or more specific manners in which the ultrasound system is to be operated to obtain images of the targets inside the phantom 600 for use by an analyzer 428 (e.g., controller) as discussed herein. For instance, the displayed instructions may instruct the technician to operate the ultrasound system 500 at one or more particular frequencies or amplitudes, for one or more particular periods of time, etc. In another arrangement, the testing routines 432 may be configured to automatically control the ultrasound system 500 (e.g., via control signals and data 505) to operate the same at one or more particular frequencies or amplitudes, for one or more periods of time, etc. (e.g., by virtue of the processor 412 loading the routines 432 into memory 416 and triggering the ultrasound system 500 to operate in such manner(s) by way of any appropriate wired or wireless connection).

In any case, the processor 412 may be configured to execute an analyzer 428 (e.g., set(s) of computer readable instructions) that is operable to analyze the digitized image signals 504 received from the ultrasound system 500 (e.g., by way of any appropriate wired or wireless connection) and reference measurement data 404 obtained from storage 420 to generate result data 408 that may be presented on the display 424 in any appropriate manner. The displayed result data 408 is configured to convey a relative level of performance of the ultrasound system 500 (e.g., of the probe 508 under test) in relation to a wide variety of operating parameters.

Broadly, the analyzer 428 may be configured to measure any appropriate characteristic data 402 (e.g., data values) from the received digitized image signals 504 and store the same in storage 420. For instance, representative types of characteristic data 402 may include pixel intensity, edge detection, image uniformity, image differential analysis, contrast and brightness, and cross-sectional comparative analyses of the images. Each respective measured characteristic data 402 may be respectively analyzed in view of corresponding reference characteristic data 404 to determine whether the measured characteristic data 402 tends to indicate that the probe 508 is functioning properly. In one arrangement, the analyzer 428 may automatically compare an absolute value of measured characteristic data 402 to an absolute value of corresponding reference characteristic data 404, where the absolute value may be associated with any appropriate tolerances such that the measured characteristic data 402 being at the absolute value or within the tolerances may tend to indicate that the measured characteristic data 402 is acceptable. As discussed herein, such comparison may be conducted on a pixel by pixel basis or on a region-by-region basis (a region being a collection of pixels), where the measured characteristic data 402 (e.g., pixel intensity, color, etc. or region metric) of each respective pixel or region of the obtained image data 504 may be compared to the corresponding reference characteristic data 404 (e.g., pixel intensity, color, etc. or region metric) of the same or related pixel or region in the reference image.

Additionally or alternatively, the analyzer 428 may automatically compare each measured characteristic data 402 to an acceptable range of values of the corresponding reference characteristic data 404 for purposes of making a determination as to the acceptability of the particular measured characteristic data 402. The disclosed tolerances and/or ranges may vary as appropriate depending on the particular type of probe 508 being utilized, the particular type of phantom 600 being utilized, other operating conditions, and/or the like. In one variation, the analyzer 428 may implement any appropriate logic or the like to indicate a degree to which the measured characteristic data 402 represents acceptable data.

In one arrangement, the analyzer 428 may measure the degree of image uniformity in the obtained image data 504 and utilize the same as a metric for overall performance analysis. In another arrangement, the analyzer 428 may correlate the degree of overall image performance to a minimum number of measured characteristic data 402 that is considered acceptable. In some situations, the analyzer 428 may utilize image convolution to match the measured characteristic data 402 to the reference characteristic data 404. For instance, performing convolution on the obtained image data 504 (or measured characteristic data 402) to more closely match a reference image (or the reference characteristic data 404) could be employed to compensate the obtained image to a point where it can be adequately compared to the reference image. In this regard, subsequent comparisons or analyses may be performed using other techniques disclosed herein.

In one embodiment, the analyzer 428 may implement feature extraction of the obtained image data 504 to obtain the measured characteristic data 402 for use in comparison to or analysis in view of the reference characteristic data 404. Specifically, such feature extraction may generally involve identifying measurable properties or characteristics to derive feature values from the obtained image data 504, where such features are intended to be more manageable, informative, and readily processed in comparative analyses. Each derived feature may be compared to corresponding reference features to determine whether the derived feature is “acceptable” and thus whether it tends to indicate the acceptability of the obtained image data 402. For instance, such feature extraction may include edge detection, image subtraction, template matching, and/or the like.

As discussed previously, the disclosed system 400 may instruct a testing technician as to the specific operating conditions under which images of the phantom 600 are to be obtained (e.g., in relation to physical orientations and attitudes between the probe 508 of the ultrasound system and the phantom 600, ultrasound frequency wavelength, ultrasound intensity, ultrasound time-domain characteristics, ultrasound frequency-domain characteristics, and signal processing methodologies). In other arrangements, however, the disclosed system 400 may provide little to no guidance to the testing technician as to any particular operating conditions for use in testing of the probe 508. In other words, once the system 400 is primed and ready to accept new images of probes under test for use in analysis, the system 400 may be configured to accept a wide variety of images of the probe under test under a wide variety of operating conditions for use in conducting an analysis of the proble 508.

For instance, the analyzer 428 may be configured to automatically determine one or more operating conditions under which the obtained image was taken and then identify a corresponding previously obtained reference image in the map having the same or similar operating conditions (e.g., set 708 of operating conditions of a particular reference image object 702 in the map 700 of FIGS. 5-6). As one simplistic example, the operating conditions of the particular obtained image may be considered the “same” as those of a particular one of the reference image objects 702 if they are within a particular percentage or range of those of the reference image object 702. As a more complex example, the various operating conditions may be assessed as part of any appropriate similarity or distance analysis to determine whether the operating conditions of the obtained image are “close enough” to the operating conditions of a particular one of the reference image objects 702 such that the operating conditions of the particular reference image object 702 are considered the same as those of the obtained image (e.g., are considered to be common operating conditions).

Upon identifying at least one corresponding reference image object 702, the analyzer may then be configured to compare the measured characteristic data 402 of the obtained images to the corresponding reference measurement data 712 of the at least one identified reference image object 702 in one or more of the manners discussed herein to determine an “acceptability” of the probe 508 (or ultrasound system 500) under test. The comparison and/or other analyses performed by the analyzer may be conducted on a pixel by pixel level, within regions (collections of multiple pixels, contiguous or non-contiguous), in relation to the images as a whole, and/or the like. During and/or upon a conclusion of any of the aforementioned analyses, the analyzer 428 may transform the results to a format appropriate for display and present the results 410 of the one or more analyses on the display 424 or the like in any appropriate manner. In one arrangement, the results may be presented in the nature of a simple “pass/fail” in relation to either each of the measured characteristic data 402 or in relation to the probe 508 as a whole. In some situations, the actual measured characteristic data 402 may not be presented on the display 424 or even made available to the technician or the like.

In some arrangements, physical placement of the probe 508 in relation to the phantom 600 for testing of the probe 508 may be dictated in any appropriate manner. For instance, one or more fixtures, markers, etc. may be included (e.g., on the phantom 600) to indicate to the operator one or more specific manners in which the probe 508 is to be positioned relative to the phantom 600 to facilitate accurate repeatability of probe testing. As discussed previously herein, the phantom in one variation may include a mechanically keyed probe-specific probe holder that will allow the rapid and precise alignment between the probe and the phantom.

It will be readily appreciated that many deviations may be made from the specific embodiments disclosed in the specification without departing from the spirit and scope of the invention. The illustrations and discussion herein have only been provided to assist the reader in understanding the various aspects of the present disclosure. For instance, while the probe 508 and console 512 have been illustrated in FIG. 4 as separate entities, the probe 508 and console 512 could also be embodied in a single entity or in multiple entities. Furthermore, one or more various combinations of the arrangements and embodiments disclosed herein are also envisioned.

Embodiments disclosed herein can be implemented as one or more software or computer program products, i.e., one or more modules of computer program instructions encoded on a computer-readable medium for execution by, or to control the operation of, data processing apparatus (processors, cores, etc.). The computer-readable medium can be a machine-readable storage device, a machine-readable storage substrate, a memory device, a composition of matter affecting a machine-readable propagated signal, or a combination of one or more of them. In addition to hardware, software that creates an execution environment for the computer program in question may be provided, e.g., software that constitutes processor firmware, a protocol stack, a database management system, an operating system, or a combination of one or more of them.

A computer program (also known as a program, software, software application, script, or code) used to provide the functionality described herein can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

While this disclosure contains many specifics, these should not be construed as limitations on the scope of the disclosure or of what may be claimed, but rather as descriptions of features specific to particular embodiments of the disclosure. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

What is claimed is:

1. A method for use in assessing a performance of an ultrasound system, the method comprising:
receiving, at a processor from an ultrasound system under test, one or more input images of a tissue mimicking phantom, wherein the one or more images contain a digital representation of the tissue mimicking phantom;
determining one or more characteristic data values from the one or more input images;
automatically identifying, by the processor, one or more corresponding respective reference data values from a database of reference data based on previously-obtained reference images of the phantom;
analyzing, by the processor, the one or more characteristic data values in view of the one or more corresponding respective reference data values;
generating, by the processor, result data based on the analyzing step, wherein the result data indicates a performance of the ultrasound system under test.

2. The method of claim 1, further including:
automatically identifying, by the processor from a database of reference images, one or more of the reference images that correspond to the one or more input images, wherein the one or more corresponding respective reference data values are indexed to the identified one or more reference images in the database.

3. The method of claim 2, wherein the one or more input images and the one or more of the reference images were obtained under common operating conditions.

4. The method of claim 3, wherein the common operating conditions include one or more of a physical orientations and attitudes between a probe of the ultrasound system and the phantom, ultrasound frequency wavelength, ultrasound intensity, ultrasound time-domain characteristics, ultrasound frequency-domain characteristics, and signal processing methodologies.

5. The method of claim 1, wherein the analyzing includes analyzing an absolute value of each of the one or more characteristic data values to an absolute value of each of the one or more corresponding respective reference data values.

6. The method of claim 5, wherein each of the one or more respective reference data values includes a tolerance, and wherein the method includes:
determining, by the processor, that a particular one of the characteristic data values is acceptable when it is within the tolerance of the corresponding respective reference data value.

7. The method of claim 1, wherein the analyzing is conducted on a pixel-by-pixel basis.

8. The method of claim 1, wherein the analyzing is conducted on a region-by-region basis, wherein each region includes a plurality of pixels.

9. The method of claim 1, wherein the analyzing is conducted on an image-by-image basis.

10. The method of claim 1, wherein each characteristic and reference data values is a pixel intensity, edge detection value, image uniformity value, contrast, and/or brightness.

11. The method of claim 1, further including:
determining, by the processor, one or more operating conditions under which the one or more input images were obtained;
automatically identifying, by the processor from a database of reference images, one or more of the reference images having one or more operating conditions that correspond to the one or more operating conditions of the one or more input images, wherein the one or more corresponding respective reference data values are indexed to the one or more of the reference images in the database.

12. A system, comprising:
a tissue mimicking phantom including a plurality of objects disposed within a tissue mimicking material;
an ultrasound system including a) an ultrasonic probe that is configured to generate and receive ultrasonic waves resulting from their interaction with the plurality of objects and b) an imaging console that is configured to process the received ultrasonic waves to generate digitized image signals;
a testing controller that is configured to process characteristic data describing the digitized image signals against corresponding characteristic data describing one or more corresponding reference image signals to generate result data indicative of a performance of the ultrasound system.
